(19) United States
(12) Patent Application Publication
(10) Pub. No.: US 2026/0174865 A1
(43) Pub. Date: Jun. 25, 2026

(54) METHOD FOR OBTAINING BIODEGRADABLE COLLOIDAL PARTICLES

(71) Applicants: UNIVERSIDAD DE GUADALAJARA, Jalisco (MX); UNIVERSIDAD DE SONORA, Sonora (MX)

(72) Inventors: Antonio TOPETE CAMACHO, Jalisco (MX); Adrián DANERI NAVARRO, Jalisco (MX); Josué Elías JUÁREZ ONOFRE, Jalisco (MX)

(21) Appl. No.: 18/877,799

(22) PCT Filed: Jun. 28, 2023
(86) PCT No.: PCT/IB2023/056702

§ 371 Date: Dec. 20, 2024

(30) Foreign Application Priority Data
Jun. 29, 2022 (MX) MX/A/2022/008128

(51) Int. Cl. A61K 47/10; A61K 9/00; A61L 27/54; C07D 471/04

(52) U.S. Cl. CPC A61K 47/10; A61K 9/0019; A61K 9/0024; A61L 27/54; C07D 471/04; A61K 2300/00

(57) ABSTRACT

The present invention relates to a system for the release of bioactive compounds comprising biodegradable colloidal particles with an average size less than 500 nm, where the biodegradable colloidal particles comprise an anionic polysaccharide and a cationic polysaccharide. Furthermore, its method of extraction includes: (a) estimating the stoichiometric charge ratio between an anionic polysaccharide, and a cationic polysaccharide; (b) preparing a solution with the anionic polysaccharide based on the stoichiometric charge ratio and adjusting to an acidic pH; (c) preparing a solution with the cationic polysaccharide based on the stoichiometric charge ratio and adjusting to an acidic pH; (d) adding the solution with the cationic polysaccharide to the solution with the anionic polysaccharide to obtain biodegradable colloidal particles; (e) reducing the particle size of the biodegradable colloidal particles; (f) separating the biodegradable colloidal particles from the supernatant; and (g) resuspending the biodegradable colloidal particles and storing them.

### FIELD OF THE INVENTION

The present invention relates to inert carriers or additives physically bound to the active ingredient, more specifically it refers to biodegradable colloidal particles and the method of extracting them.

### BACKGROUND OF THE INVENTION

Drug delivery systems are now as important as the drug itself. Controlled release provides prolonged administration of a drug while maintaining its blood concentration within the therapeutic limits. Drug delivery systems can thus influence pharmacological activity by modulating its release from the vehicle. Other advantages include increased patient compliance (given a reduction in dosing frequency), noninvasive routes of administration, minimized local and systemic side effects, and therefore a reduced toxicity profile. Nanosized drug delivery systems can deliver drugs to the site of action in a pre-designed manner, thereby minimizing side effects and improving the bioavailability of that drug. For example, colloidal drug carriers, such as micelles, liposomes, nanoparticles, and emulsions, are used to increase drug concentrations that pass through the blood-brain barrier into the brain. Furthermore, the active ingredients entrapped within the drug carrier system may be protected against enzymatic degradation.

The ability of these systems to cross external barriers and access the interior of the organism depends on both their size and their composition. Nanosized particles increase the degree of transport compared to larger particles. Furthermore, if they are prepared from naturally occurring and biocompatible polymers, the chances of them being transported naturally through the body's mucous membranes, by known transport mechanisms and without altering epithelial physiology, increase. However, the main limitations of nanoparticles prepared from synthetic polymers are the excessive costs incurred and the production technologies inconvenient for industrial scaling, the use of toxic solvents during the production process and the leakage of drugs before reaching the target regions. In order to overcome all limitations, research has focused on discovering new methods for obtaining novel biodegradable nanoparticles from naturally occurring and biocompatible polymers.

For example, Katuwavila et al., in his publication “Chitosan-Alginate Nanoparticle System Efficiently Delivers Doxorubicin to MCF-7 Cells, Journal of Nanomaterials”, describe a method for obtaining alginate-chitosan-DOX nanoparticles through a process of dropwise addition of a mixture of chitosan (2 mg/mL, pH 4.8) and the surfactant Tween 80 to an alginate solution (1 mg/mL, pH 5.2) previously mixed with doxorubicin (DOX). Although both chitosan and alginate have been reported to be pharmaceutically acceptable natural polymers for targeted drug delivery, their use individually or together involves the use of surfactants, chemical cross-linking agents and/or organic solvents that can modify the properties of the active ingredient to be administered, even increasing production costs.

Therefore, there is a need to find new methods for obtaining active ingredient release systems that allow efficient incorporation into the biological system, and rapid biodegradability of the particles in the biological medium. Furthermore, these systems should be easily produced and stable during storage and transport.

### OBJECTS OF THE INVENTION

Considering the defects of the prior art, it is an object of the present invention to provide a system for the release of bioactive compounds comprising colloidal particles based on biocompatible and biodegradable polymers.

Likewise, another object of the present invention relates to a method for obtaining biodegradable colloidal particles.

These and other objects are achieved by biodegradable colloidal particles in accordance with the present invention.

### SUMMARY OF THE INVENTION

To this end, a first aspect of the present invention relates to a system for the release of bioactive compounds, comprising biodegradable colloidal particles which in turn comprise an anionic polysaccharide, a cationic polysaccharide, and at least one bioactive compound, which have an average size of less than 500 nm.

A second aspect of the present invention relates to a method for obtaining biodegradable colloidal particles, which comprises the steps of: (a) estimating the stoichiometric charge ratio between an anionic polysaccharide based on its degree of deesterification, and a cationic polysaccharide based on its degree of deacetylation; (b) preparing a solution with the anionic polysaccharide based on the stoichiometric charge ratio and adjusting to an acidic pH; (c) preparing a solution with the cationic polysaccharide based on the stoichiometric charge ratio and adjusting to an acidic pH; (d) adding the solution with the cationic polysaccharide to the solution with the anionic polysaccharide to obtain biodegradable colloidal particles; (e) reducing the particle size of the biodegradable colloidal particles; (f) separating the biodegradable colloidal particles from the supernatant; and (g) resuspending the biodegradable colloidal particles and storing them.

### BRIEF DESCRIPTION OF THE DRAWINGS

The novel aspects which are considered characteristic of the present invention will be set forth with particularity in the appended claims. However, its features and advantages will be better understood in the examples, when read in relation to the attached figures, where:

FIG. 1 shows a FTIR-ATR spectrophotometry curve of chitosan with a resolution of 0.5 cm⁻¹. The peaks at 1320 and 1420 cm⁻¹ are indicated by arrows, and they correspond to the N-acetyl and N-amino groups, respectively.

FIG. 2 shows the results of hydrodynamic diameter in nanometers (DH, intensity averaged) for the biodegradable chitosan/alginate colloidal particles prepared with different charge ratios.

FIG. 3 shows the Zeta potential obtained for the biodegradable chitosan/alginate colloidal particles prepared with different charge ratios.

FIG. 4 shows biodegradable chitosan/alginate colloidal particles prepared with different charge ratios analyzed by atomic force microscopy (AFM).

FIG. 5 shows the results of the hydrodynamic diameter in nanometers (DH, intensity averaged) obtained for biodegradable chitosan/alginate colloidal particles subjected to different pH values.

FIG. 6 shows the results of the polydispersity index (PDI) obtained for the biodegradable chitosan/alginate colloidal particles subjected to different pH values.

FIG. 7 shows the Zeta potential results obtained for the biodegradable chitosan/alginate colloidal particles subjected to different pH values.

FIG. 8 shows the hydrodynamic diameter in nanometers (DH, intensity averaged) for biodegradable chitosan/alginate colloidal particles obtained using different surfactants and experimental conditions.

FIG. 9 shows the mass of DOX-HCl encapsulated in biodegradable chitosan/alginate colloidal particles with different charge ratios.

FIG. 10 shows the encapsulation efficiency of DOX-HCl (% EE) in biodegradable chitosan/alginate colloidal particles with different charge ratios.

FIG. 11 shows the mass of ICG encapsulated in biodegradable chitosan/alginate colloidal particles with different charge ratios.

FIG. 12 shows the encapsulation efficiency of ICG (% EE) in biodegradable chitosan/alginate colloidal particles with different charge ratios.

FIG. 13 shows the heating curve of ICG-functionalized chitosan/alginate biodegradable colloidal particles, where DT expresses the recorded temperature difference. The laser power was 2 W. The line corresponds to the fit to the Roper model and the points to the experimental data.

FIG. 14 shows the results of atomic force microscopy (AMF) performed on the biodegradable oligochitosan/alginate colloidal particles obtained in test 1 in a scanning area of 2500 nm×2500 nm (left) and 5000 nm×5000 nm (right). The particles have an intensity-averaged hydrodynamic diameter of 339.0 nm and a Zeta Potential=−29.4 mV.

FIG. 15 shows the results of atomic force microscopy (AMF) performed on the biodegradable oligochitosan/alginate colloidal particles obtained in test 2 in a scanning area of 2500 nm×2500 nm (left) and 5000 nm×5000 nm (right). The particles have an intensity-averaged hydrodynamic diameter of 358.5 nm and a Zeta Potential=−41.4 mV.

FIG. 16 shows the results of atomic force microscopy (AMF) performed on the biodegradable oligochitosan/alginate colloidal particles obtained in test 3. The particles have an intensity-averaged hydrodynamic diameter of 346.0 nm and Zeta Potential=−39.5 mV.

FIG. 17 shows a calibration curve of DOX-HCl obtained from a UV-vis spectrophotometer where the equation was obtained: y=28.588x+0.0145 with an R²=0.99.

FIG. 18 shows a comparison of the encapsulation efficiency (% EE) obtained with two different methods for the functionalization of DOX-HCl in biodegradable oligochitosan/alginate colloidal particles.

FIG. 19 shows an ICG calibration curve obtained from a UV-vis spectrophotometer where the equation was obtained: y=39.704x+0.105 with an R²=0.99.

FIG. 20 shows a comparison of the encapsulation efficiency (% EE) and the percentage loading obtained with the two methods for the functionalization of ICG in biodegradable oligochitosan/alginate colloidal particles.

### DETAILED DESCRIPTION OF THE INVENTION

The present invention exhibits certain advantages over the state of the art, among which we can mention that the system for the release of bioactive compounds, which comprises biodegradable colloidal particles, allows a safe incorporation into the biological system and rapid biodegradability due to the natural characteristics of the polysaccharides used during its preparation. In addition, its method of extraction does not require the use of surfactants, chemical cross-linking agents and/or organic solvents, which facilitates their production and ensures their stability during storage and transport.

Therefore, the present invention relates first of all to a system for the release of bioactive compounds, comprising biodegradable colloidal particles which in turn comprise an anionic polysaccharide, a cationic polysaccharide, and at least one bioactive compound; which have an average size of less than 500 nm.

Preferably, the anionic polysaccharide is alginate. The cationic polysaccharide is selected from chitosan, oligochitosan or mixtures.

More preferably, the cationic polysaccharide is oligochitosan.

The anionic polysaccharide and the cationic polysaccharide are joined by electrostatic attraction.

Preferably, the bioactive compounds have antitumor activity and are photosensitizing.

A second aspect of the present invention relates to a method for obtaining biodegradable colloidal particles, which comprises the steps of: (a) estimating the stoichiometric charge ratio between an anionic polysaccharide based on its degree of deesterification, and a cationic polysaccharide based on its degree of deacetylation; (b) preparing a solution with the anionic polysaccharide based on the stoichiometric charge ratio and adjusting to an acidic pH; (c) preparing a solution with the cationic polysaccharide based on the stoichiometric charge ratio and adjusting to an acidic pH; (d) adding the solution with the cationic polysaccharide to the solution with the anionic polysaccharide to obtain biodegradable colloidal particles; (e) reducing the particle size of the biodegradable colloidal particles; (f) separating the biodegradable colloidal particles from the supernatant; and (g) resuspending the biodegradable colloidal particles and storing them.

Preferably, the anionic polysaccharide is alginate.

Preferably, the cationic polysaccharide is selected from chitosan, oligochitosan or mixtures. More preferably, the cationic polysaccharide is oligochitosan.

Preferably, the solvent for preparing polysaccharide solutions is water.

Preferably, the degree of deesterification in step (a) is determined by potentiometric or conductometric titration.

Preferably, the degree of deacetylation in step (a) is determined by nuclear magnetic resonance (NMR), UV-vis spectrophotometry, potentiometric or conductometric titration, or FTIR-ATR spectrophotometry.

Preferably, the anionic polysaccharide solution is adjusted to a pH of 4.

Preferably, the cationic polysaccharide solution is adjusted to a pH of 5.

Preferably, the cationic polysaccharide solution is filtered before use in step (d).

Preferably, step (d) is performed by a constant drip technique with vigorous stirring.

Preferably, the particle size reduction in step (e) is accomplished by sonicating the biodegradable colloidal particles by placing the particles in a vessel containing ice water to mitigate overheating of the particles for periods of between 5 and 10 minutes.

Preferably, the separation in step (f) is carried out by centrifugation at a speed of between 6000 and 7000 rpm.

Preferably, resuspension in step (g) is performed by vortexing for at least 10 minutes.

Preferably, the method comprises an additional step of functionalizing the biodegradable colloidal particles with at least one bioactive compound.

Preferably, the functionalization step is carried out prior to step (d), where the solution of the cationic polysaccharide obtained in step (e) is mixed with a solution with at least one bioactive compound.

Preferably, the functionalization step is performed after step (g), which in turn comprises the steps of: (i) adding a solution with at least one bioactive compound to a container with the biodegradable colloidal particles; (ii) adding deionized water to the mixture; (iii) stirring; (iv) centrifuging; (v) resuspending the previously functionalized biodegradable colloidal particles with deionized water to eliminate the non-trapped bioactive compound; and (vi) lyophilizing the functionalized biodegradable colloidal particles.

More preferably, step (iii) stirring is performed on a vortex mixer for at least 1 hour.

More preferably, step (iv) centrifugation is performed for between 6000 and 7000 rpm for between 15 and 30 minutes at a temperature of at least 10° C.

More preferably, the bioactive compounds have antitumor activity and are photosensitizing.

The term “photosensitizer” refers to a bioactive compound used for photodynamic therapy.

It refers to that which, if present in the body, can cause a skin reaction by interaction with ultraviolet radiation, that is, it sensitizes to sunlight.

The advantages of the present invention will be better understood from the following examples, which are presented solely for illustrative purposes to allow a full understanding of the preferred embodiments of the present invention, without implying that there are no other embodiments not illustrated that can be put into practice based on the detailed description given above.

### Example 1

An essay was carried out to exemplify the method for obtaining biodegradable colloidal particles from polysaccharides, particularly chitosan and alginate, in accordance with the principles of the present invention.

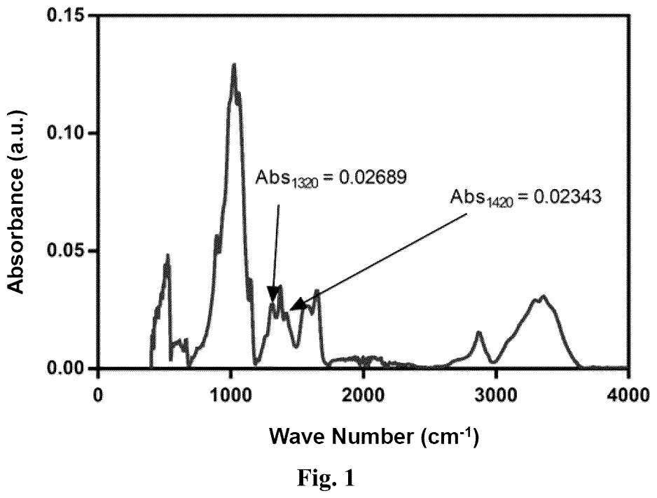

First, in order to determine the charge ratios for the production of the particles, 100% deesterification of the alginate was considered, data that was subsequently corrected. The density of negative charges for alginate (Cat: A1112) using this assumption is d₋=5.16×⁻³ moles of negative charges per gram of alginate. Then, the degree of deacetylation (% DD) of the low molecular weight chitosan (Mw˜125000 g/mol) was determined in order to determine the charge ratio for the preparation of biodegradable colloidal particles. The % DD indicates the molar percentage of glucosamine monomeric units with respect to the total monomeric groups, that is, glucosamine units plus N-acetylglucosamine units. Thus, % DD is translated as the concentration of —NH groups₂ per unit mass of chitosan. There are several techniques to determine % DD, including NMR, UV-vis spectrophotometry, potentiometric and conductometric titration, and FTIR-ATR spectrophotometry. In this example, FTIR-ATR spectrophotometry was used because of its speed, accuracy and simplicity. Approximately 500 mg of chitosan powder (cat. 448869-250G) in the ATR optical reader and the spectra were taken with a resolution of 0.5 cm⁻¹, performing 30 repetitions to increase the definition of the peaks, the results are shown in FIG. 1. The baseline subtraction of the spectra was subsequently performed using Origin 8.0® software, and the absorbance was determined at 1320 and 1420 cm⁻¹, corresponding to the proportion of the N-acetyl and N-amino groups, respectively.

The % DD was obtained from the degree of acetylation (% DA) using the equation:

$$DA ( \% ) = ( 31.92 \frac{{A}_{1320}}{{A}_{1420}} ) - 12.20$$

For chitosan, absorbances of 0.02689 and 0.02343 were obtained at 1320 and 1420 nm, respectively, therefore, % DA=24.43% and % DD=75.57%, within the range reported by the supplier (% DA≥75%). Taking the average molecular weight based on viscosity provided by the supplier Pm=120000 g/mol, it was estimated that the density of positive charges in chitosan is d₊=3.97×10⁻³ moles of positive charges per gram of chitosan.

The relationship between the negative and positive charges (R) of each of the alginate/chitosan mixtures was determined using the following equation:

$$R = \frac{{V}_{A} \times {C}_{A} \times d \_}{{V}_{Q} \times {C}_{Q} \times {d}_{+}}$$

Where V_{A} is the volume of alginate solution, C_{A} is the mass concentration of the alginate solution and d₋ is the number of negative charges per gram of alginate. The same definitions apply for the variables in the denominator of the relationship, but for chitosan.

Once the charge ratio was established, solutions of the polysaccharides were prepared and the pH was adjusted, the alginate was adjusted to a pH=4 and the chitosan was adjusted to a pH=5, before mixing them. The solutions were filtered with a 0.45-micron filter, in order to eliminate impurities.

For the preparation of colloidal particles, a method of mixing by dripping from one solution to the other was followed, where the dripping was constant and the stirring vigorous. After mixing the biopolymers, the mixture is subjected to a sonication process for periods of 5 to 10 minutes with a probe (Fisherbrand™ Model 120 Sonic Dismembrator), where the container (50 mL conical tubes or 20 mL glass vials) containing the mixture was placed in an ice water bath to prevent overheating of the sample and possible damage to the bioactive compounds that are sought to be encapsulated. Finally, it was washed by centrifugation. The generated colloidal particles were centrifuged at 6000 rpm, the supernatant was recovered, and the sediment was resuspended by vortex.

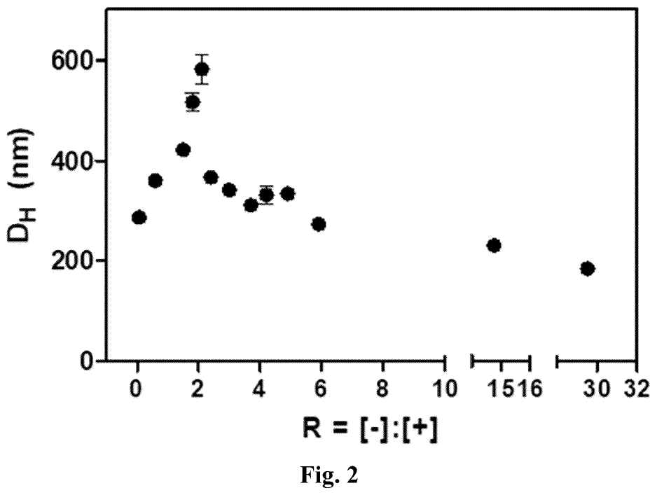

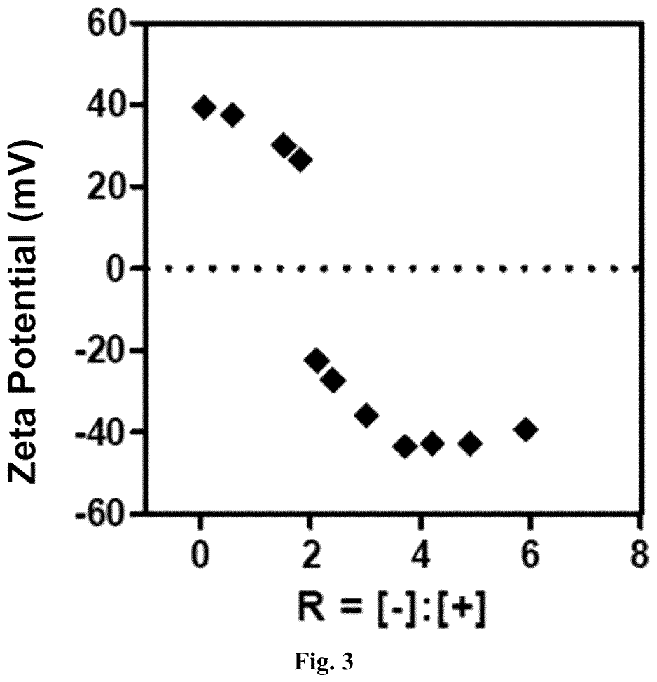

FIG. 2 shows the results of hydrodynamic diameter in nanometers (DH, intensity averaged) and FIG. 3 shows the Zeta potential obtained for preparations with different charge ratios (these ratios are estimated, since 100% deesterification of the alginate is assumed and an average molecular weight provided by the supplier is used). In the developed method, the alginate solution was placed as a “receiver”, that is, it was kept under constant agitation and the chitosan was dripped onto it. As can be seen, the hydrodynamic diameter decreases with an increasing charge ratio (R), obtaining hydrodynamic diameters of up to 184.4±1.3 nm with the ratio R=30. As seen in FIG. 3, around a ratio close to 1, a transition from positive zeta potential values to negative values occurs. That is, the effective isoelectric point of colloidal particles lies around this ratio. The above coincides with the particle size data, since when approaching a value of R=1, an increase in size was observed, due to the decrease in electrostatic stability and the aggregation of the particles.

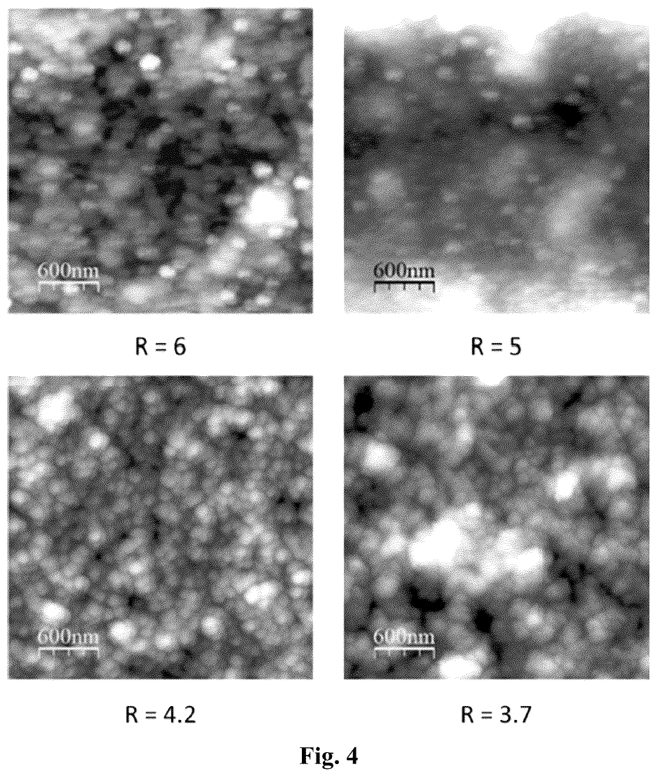

Some of the samples were analyzed by atomic force microscopy (AFM) to obtain a diameter distribution, which is shown in FIG. 4. For example, for the sample with an R=4.2, an average diameter of 135±18 nm was obtained. The average was obtained by analyzing the AFM image with ImageJ® software. In colloidal particles obtained from polysaccharides with high water absorption capacity, the diameter obtained shown in FIG. 4 is smaller than the hydrodynamic diameter obtained using the dynamic light scattering technique (Zetasizer).

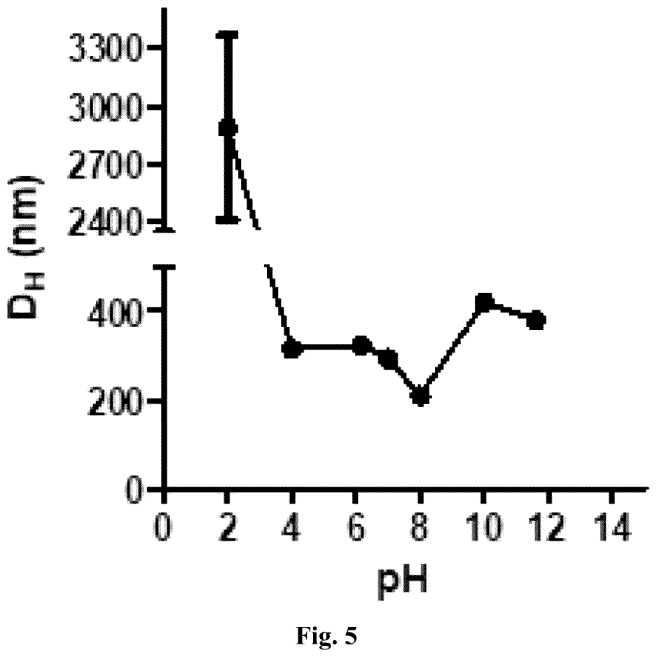

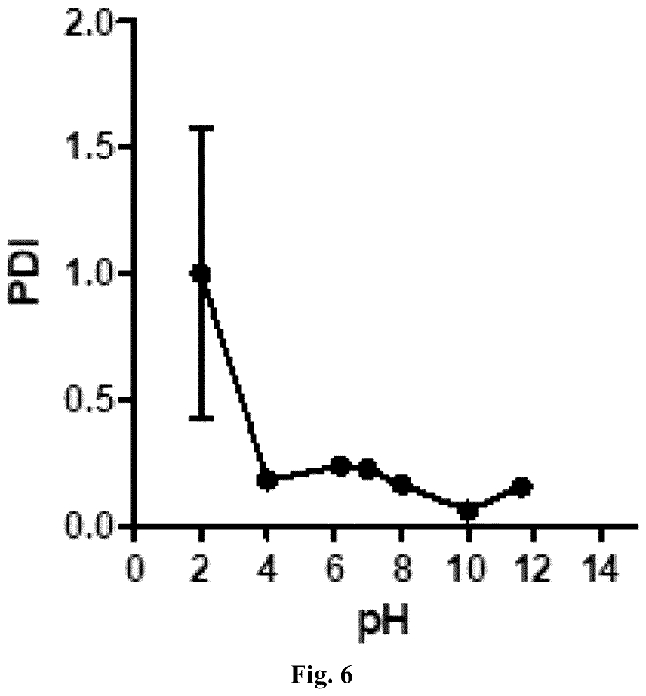

To evaluate the colloidal stability of the particles (R=3) at different pH values, samples were prepared by mixing 1 mL of particles with 1 mL of 50 mM NaCl and the pH was adjusted to different values with HCl or NaOH. The hydrodynamic diameter, polydispersity index (PDI) and Zeta potential were measured at different pH values. The results obtained are shown in FIGS. 5, 6 and 7, respectively. It is shown that the hydrodynamic diameter remains stable in the pH range of 4 to 7, even presenting a slight decrease in size at pH=8, attributable to a reconfiguration of the alginate and chitosan polymer chains. At more alkaline pH values, 10 and 12, a small increase in the hydrodynamic diameter is observed, without reaching a drastic destabilization of the colloidal suspension. On the contrary, at pH=2, a destabilization of the colloidal particles was observed, manifested by the substantial increase in diameter to values greater than 2700 nm. This destabilization is due to the protonation of the alginate carboxyl groups, that is, to a decrease in the magnitude of the Z potential value which results in coalescence and sedimentation of the particles.

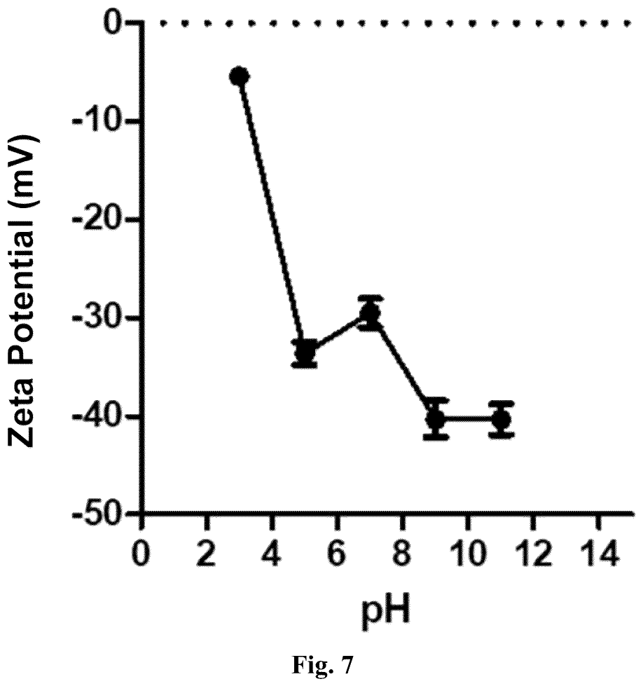

This behavior allows us to infer that the configuration or structure of the particles with this composition could be an internal region or core rich in chitosan chains surrounded by a crown with a high concentration of alginate. Since the particles are at a pH greater than 3 (pKa of sodium alginate is between 3.3 and 3.6), they are stabilized by electrostatic repulsion of the double electrical layer they possess. When the pH decreases below the pKa of the alginate, the carboxyl groups are protonated, the colloidal particles losing their negative charge as shown in FIG. 7, going from Zeta potential values less than −30 mV to a value of −5 mV, very close to neutrality, triggering the aggregation of the particles.

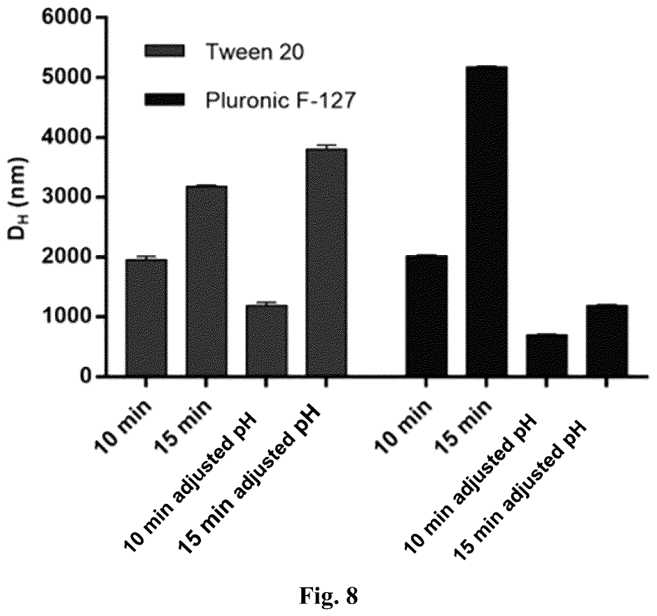

During the development of the method for preparing colloidal particles, different strategies were tested. The objective was to obtain sizes smaller than 1000 nm. Initially, attempts were made to prepare them by dropwise addition of an alginate solution to a chitosan solution without sonicating them and without adjusting the pH of the precursor solutions. The result was the formation of gels larger than 1 mm, visible to the naked eye as precipitates. At this point it was decided to follow a drip addition method using an infusion pump, sonicating simultaneously. The result was a decrease in size, but still above 1000 nm. Reviewing the available literature, it was found that some authors had used stabilizers to control the size of micro- and nanogels. This strategy was attempted and particles with hydrodynamic diameters around 800 nm were obtained as shown in FIG. 8. Particles prepared with solutions adjusting the pH value, 10 minutes of sonication and using Pluronic F127 (stabilizer) were the smallest. Subsequently, it was decided to filter the chitosan solutions before mixing them with the alginate by dripping.

This filtration was intended to remove large visible aggregates, as well as other impurities. The solution was filtered with a 0.45-micron filter, obtaining a clear, aggregate-free, and slightly viscous solution. What could also have happened when performing this filtration is that large polymer chains of chitosan were removed. Thus, when the chitosan solution was filtered, the larger chains were most likely eliminated, which was reflected in the production of particles with average diameters less than 500 nm.

### Example 2

An assay was performed where biodegradable colloidal particles from natural biopolymers obtained in example 1 were functionalized with doxorubicin hydrochloride (DOX-HCl) and indocyanine green (ICG) separately, with the purpose of measuring their functionality as delivery and release systems for bioactive compounds.

Due to their physicochemical properties, particularly their high density of positive and negative charges and their gel-like structure, biodegradable colloidal particles of alginate and chitosan have a high capacity to encapsulate and/or trap bioactive compounds with positive and negative charges. To evaluate the ability to trap antineoplastic and photosensitizing drugs, the particles were incubated with doxorubicin hydrochloride (DOX-HCl) and indocyanine green (ICG) solutions separately and the amount of trapped drug was quantified by comparing a control sample with deionized water without particles.

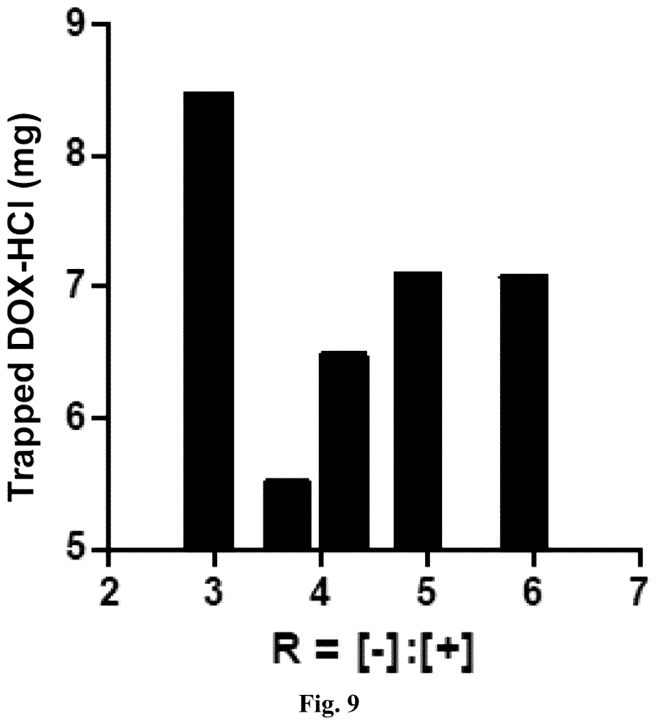

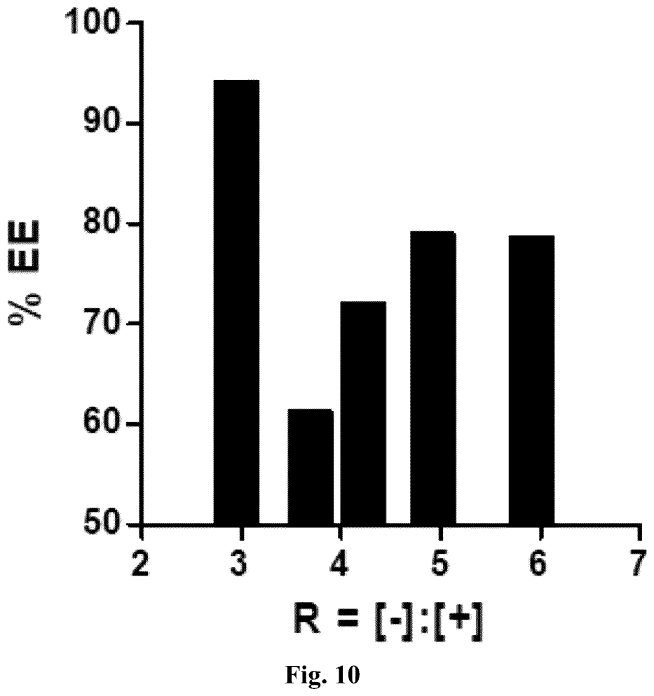

Colloidal particles of alginate and chitosan with charge ratio values >2 and Zeta potentials <−20 mV, entrapped high amounts of DOX-HCl by incubation for 24 h at room temperature and moderate shaking. FIGS. 9 and 10 show the amounts of DOX-HCl and encapsulation efficiency (% EE) for particles with different charge ratios.

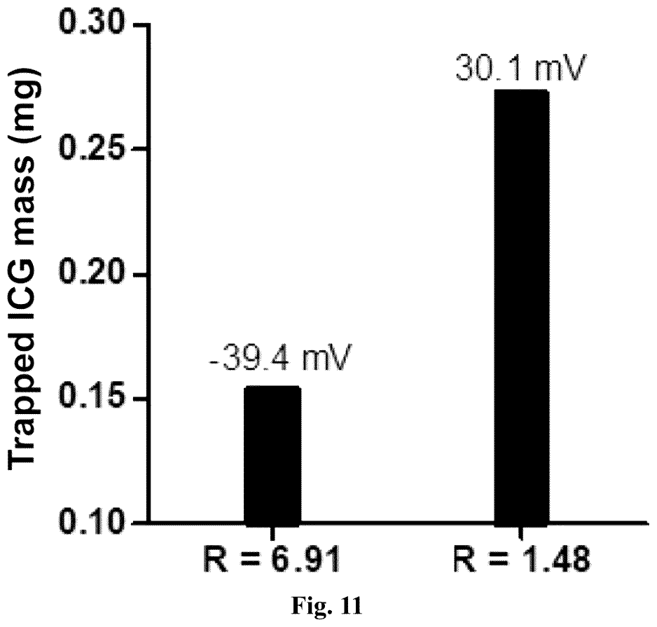

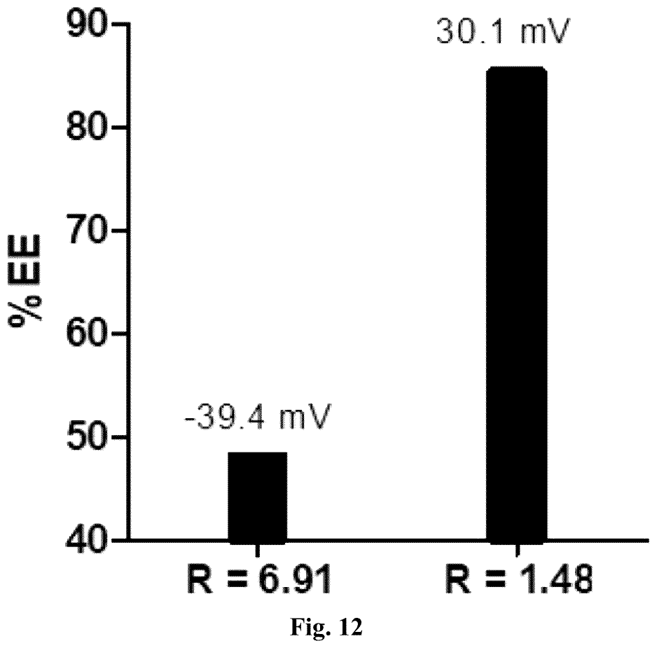

Similarly, a preliminary ICG charge test was performed on a particle sample with R=7 and Zeta potential=−39.4 mV; and on another sample with R=1.5 and Zeta potential=+30.1 mV. Samples were prepared by adding 50 μL of a 12.9 mM ICG solution to 1 mL of colloidal particles. A control sample was prepared with the same amount of ICG, i.e., 50 μL of a 12.9 mM ICG solution, added to a volume of 1 mL of deionized water. Two samples were chosen with Zeta potential of similar magnitude, but different charges, that is, one sample with a negative surface charge and another with a positive surface charge. FIGS. 11 and 12 show the ICG amounts and encapsulation efficiency (% EE) for particles with different charge ratios. It was observed that the sample with R=1.5 and Zeta potential=+30.1 mV trapped 1.8 times more ICG than the negatively charged sample. This is due to the negative charge that ICG has due to the ionized sulfonate groups and its electrostatic interaction with the positive charges of the colloidal particles. Colloidal particles with ICG can act as systems for photodynamic and photothermal therapy (phototherapies). Preliminary results of the determination of the photothermal properties of the particles are presented below, particularly the determination of their photothermal efficiency by fitting the temperature rise data as a function of irradiation time with a laser of 800 nm wavelength. The data were fitted to the model reported by Roper et al. Transfer Transduced by Surface Plasmon Resonant Gold Nanoparticles, The Journal of Physical Chemistry C 111(9) (2007) 3636-3641; and following the methodology reported by Almada et al. Photothermal conversion efficiency and cytotoxic effect of gold nanorods stabilized with chitosan, alginate and poly(vinyl alcohol), Materials Science and Engineering: C 77 (2017) 583-593.

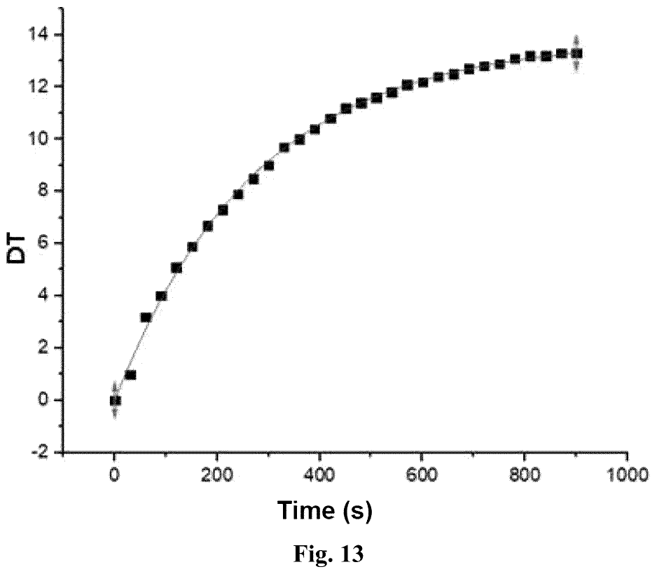

The photothermal efficiency was measured by adding 2 mL of the ICG particles to a quartz cell and stirring the solution to mix the sample and induce a homogeneous temperature increase. The samples were irradiated with a laser of 800 nm wavelength, at different powers (1, 1.5 and 2 W) for periods of 15 minutes and then 5 minutes without irradiation. The heating curve using 2 W irradiation is shown in FIG. 13 below. The temperature was measured with a K-type thermocouple connected to the digital thermometer and the temperature was recorded every 30 seconds. The quartz cell was insulated to prevent evaporation of the solution and heat dissipation.

The same treatment was done with water to obtain the parameter₀, which was calculated by irradiating water with resistivity 18.2 MΩ for 15 minutes at the same powers as the biodegradable colloidal particles. In addition, biodegradable colloidal particles without ICG were also measured to compare their photothermal effect. Analyzing the characteristic rate constant tₛ, it is observed that the greatest increase in temperature occurs in the first 4 to 5 minutes of irradiation, then the rate of increase decreases, as seen in FIG. 13. The presence of ICG in biodegradable colloidal particles gave them photoresponsive properties, dependent on the intensity of the light stimulus and the concentration of the compound in the particles. The particles with ICG show a considerable increase in temperature, the maximum thermal efficiency of the particles was 50.68% and the ΔTₘₐₓ obtained was 13.3 degrees. The thermal efficiency turned out to be strongly dependent on the irradiation power, moreover, it is possible to control the temperature increase by the combined variation of the number of particles per unit volume and the laser power.

### Example 3

An essay was carried out to exemplify the method for obtaining biodegradable colloidal particles from polysaccharides, particularly oligochitosan and alginate, in accordance with the principles of the present invention.

Based on the results obtained with low molecular weight chitosan (Mw˜125000 g/mol), whose solution was passed through a 0.45-micron filter, an attempt was made to obtain similar results using an oligochitosan (Mw˜5000 g/mol), attempting to have the same effect, but without having to filter through such a small pore size, reducing efforts and costs. Particles were prepared with three different volumetric ratios of oligochitosan and alginate solutions: 2.5 mL of alginate and 5 mL of oligochitosan (Trial 1), 5 mL of oligochitosan and 2.5 mL of alginate (Trial 2) and 5 mL of alginate and 5 mL of oligochitosan (Trial 3). After adding the oligochitosan solution dropwise to the alginate solution, all samples were sonicated for 5 minutes at 80% amplitude in an ice bath. For atomic force microscopy (AFM) imaging, samples were centrifuged at 9000 rpm at 10° C. for 30 minutes. The supernatant was discarded, and the samples were resuspended by pipetting and then sonication in a bath for 2 minutes.

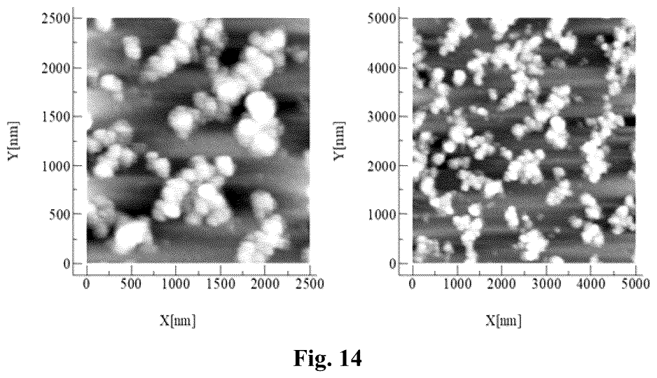

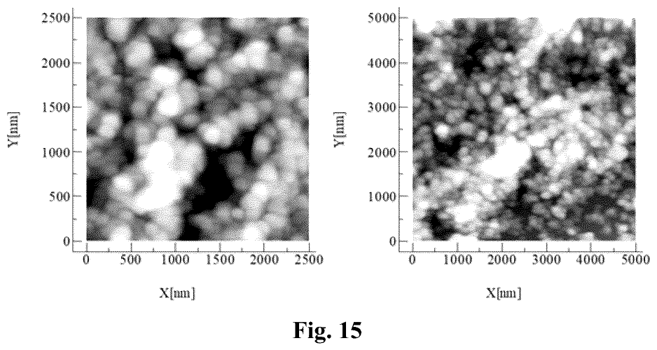

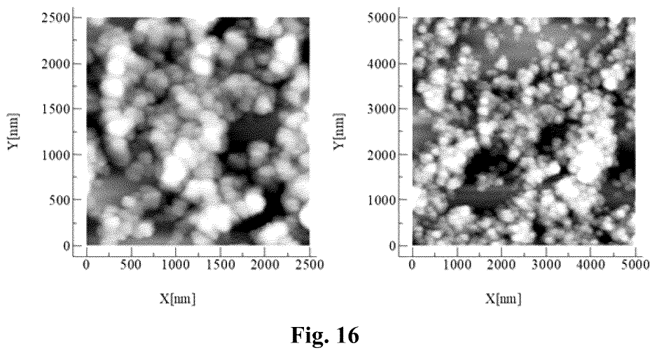

FIG. 14 shows the results of atomic force microscopy (AMF) performed on the biodegradable oligochitosan/alginate colloidal particles obtained in test 1 in a scanning area of 2500 nm×2500 nm (left) and 5000 nm×5000 nm (right), where the particles presented an intensity-averaged hydrodynamic diameter of 339.0 nm and a Zeta Potential=−29.4 mV. On the other hand, FIG. 15 shows the results of atomic force microscopy (AMF) performed on the biodegradable oligochitosan/alginate colloidal particles obtained in test 2 in a scanning area of 2500 nm×2500 nm (left) and 5000 nm×5000 nm (right), where the particles presented an intensity-averaged hydrodynamic diameter of 358.5 nm and a Zeta Potential=−41.4 mV. Finally, FIG. 16 shows the results of atomic force microscopy (AMF) performed on the biodegradable oligochitosan/alginate colloidal particles obtained in test 3, in a scanning area of 2500 nm×2500 nm (left) and 5000 nm×5000 nm (right), where the particles presented an intensity-averaged hydrodynamic diameter of 346.0 nm and Zeta Potential=−39.5 mV.

### Example 4

An experiment was carried out where biodegradable colloidal particles were functionalized from natural alginate/oligochitosan polysaccharides (oligochitosan molecular weight=5000 g/mol) with doxorubicin hydrochloride (DOX-HCl), using two different methods: (i) functionalization of previously prepared biodegradable colloidal particles with DOX-HCl; and (ii) functionalization with DOX-HCl during the gelation process of the biodegradable colloidal particles. The above, with the purpose of evaluating their functionality as systems for administration and release of bioactive compounds.

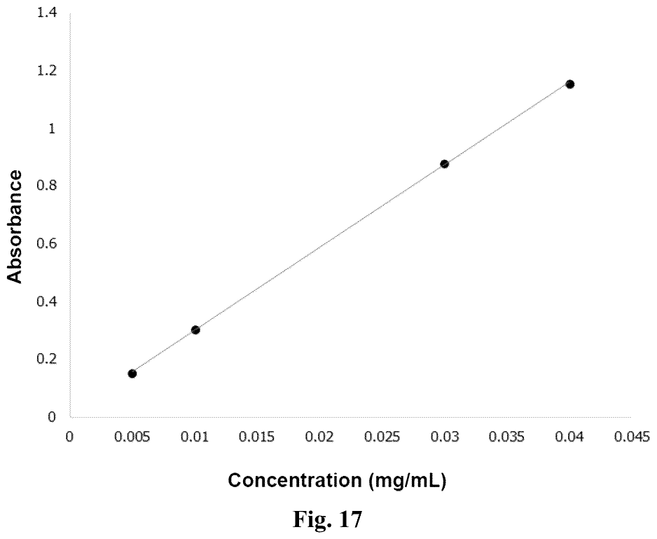

In the first method (i), particles were prepared with the volumetric ratio of 5 mL of alginate (1.5 mg/mL) with 5 mL of oligochitosan (1 mg/mL). The preparation and washing method previously described was followed. Once the particles were washed, the following protocol was followed: (i) 0.5 mL of the particles were placed in 1.5 mL conical tubes; (ii) A volume of 10, 20, 30, 40 or 50 μL of a DOX-HCl solution (1 mg/mL) was added; (iii) The necessary amount of deionized (DI) water was added to each tube to complete a total volume of 1 mL; (iv) They were left stirring in a vortex mixer for 1 hour; (v) They were centrifuged at 9000 rpm for 30 min at 10° C.; (vi) The supernatant was removed and the amount of untrapped DOX-HCl was determined by a calibration curve obtained in a UV-vis spectrophotometer shown in FIG. 17, where the equation was obtained: y=28.588x+0.0145 with an R²=0.99; (v) The mass of entrapped DOX-HCl, the encapsulation efficiency (% EE) and the encapsulation percentage of DOX-HCl were determined for each sample. Table 1 shows the results of DOX-HCl encapsulation in alginate/oligochitosan particles.

TABLE 1

| | Mass of | | |
|---|---|---|---|
| | entrapped DOX-HCl | | % DOX-HCl |
| Sample | (mg) | % EE | (theoretical) |
| 10 | 0.0071 | 70.2 | 1.12 |
| 20 | 0.013983 | 69.9 | 2.37 |
| 30 | 0.022112 | 73.7 | 3.53 |
| 40 | 0.033605 | 84.0 | 5.37 |
| 50 | 0.046558 | 93.1 | 7.45 |

In the second method (ii) during the gelation process a DOX-HCl solution was mixed with the alginate solution and subsequently the oligochitosan was added as previously described. It was carried out according to the following method: (i) 100 μL of DOX-HCl solution (1 mg/mL) was added to 5 mL of alginate (1.5 mg/mL); (ii) Oligochitosan solution (1 mg/mL) was added dropwise into alginate solution; (iv) Particles were centrifuged at 9000 rpm for 30 min at 10° C.; (v) The concentration of DOX-HCl in the supernatant was determined and therefore the mass of untrapped DOX-HCl; (vi) The mass of entrapped DOX-HCl, % EE and % DOX-HCl were calculated. Table 2 shows the results obtained from the encapsulation of DOX-HCl in the alginate/oligochitosan particles.

TABLE 2

| Mass of | | |
|---|---|---|
| entrapped DOX-HCl | | % DOX-HCl |
| (mg) | % EE | (theoretical) |
| 0.0423 | 42.3 | 3.42 |

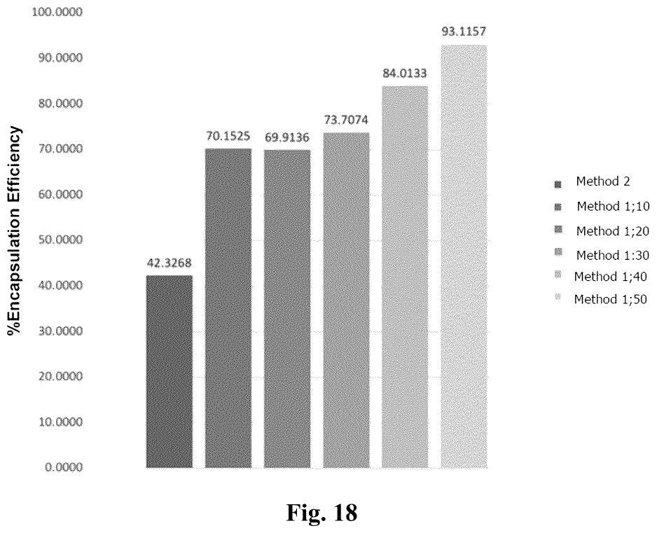

FIG. 18 shows a comparison of the encapsulation efficiency (% EE) obtained with the two methods for DOX-HCl functionalization of biodegradable oligochitosan/alginate colloidal particles. It is important to emphasize that, although in the first method (i) higher percentages of encapsulation efficiency and trapped DOX-HCl mass were obtained, the release kinetics are most likely very different in the samples prepared by the two methods. A much faster release would be expected in the samples prepared by the first method (i), since DOX-HCl is probably adsorbed on the surface, while the release could be slower in the sample loaded with the second method (ii), where DOX-HCl would be expected to be in more internal regions of the particles.

### Example 5

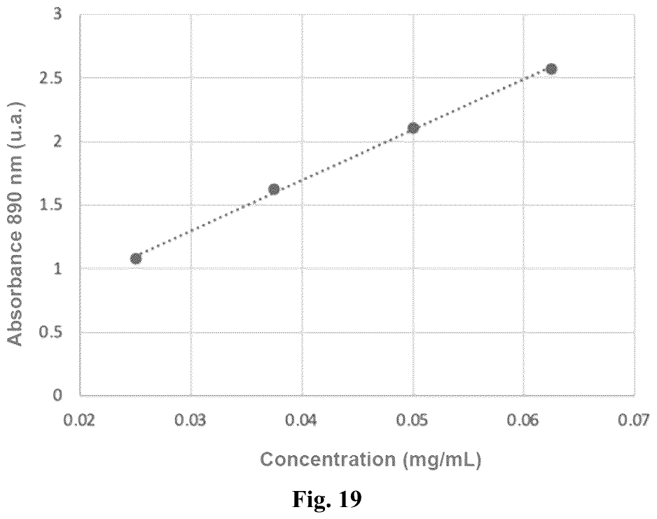

An experiment was carried out where biodegradable colloidal particles from natural alginate/oligochitosan polysaccharides (oligochitosan molecular weight=5000 g/mol) were functionalized with indocyanine green (ICG), using two different methods: (i) ICG functionalization with previously prepared biodegradable colloidal particles; and (ii) ICG functionalization in the gelation process of the biodegradable colloidal particles. The above with the purpose of evaluating their functionality as systems for administration and release of bioactive compounds. In the first method of ICG adsorption on previously prepared particles, biodegradable colloidal particles were prepared with the volumetric ratio of 2.5 mL of alginate (1.5 mg/mL) with 5 mL of oligochitosan (1 mg/mL). The preparation and washing method previously described was followed. Once the particles were washed, the following protocol was followed: (i) 0.5 mL of the biodegradable colloidal particles were placed in 1.5 mL conical tubes; (ii) A volume of 10, 20, 30, 40 or 50 μL of an ICG solution (5 mg/mL) was added; (iii) The necessary amount of deionized (DI) water was added to each tube to complete a total volume of 2 mL; (iv) They were left stirring in a vortex mixer for 1 hour; (v) They were centrifuged at 15000 rpm for 15 min at 10° C.; (vi) The supernatant was removed and the amount of untrapped ICG was determined by a calibration curve in a UV-vis spectrophotometer shown in FIG. 19, where the equation was obtained: y=39.704x+0.105 with an R²=0.99; (vii) The mass of entrapped ICG, encapsulation efficiency (% EE) and percentage of ICG encapsulation (% ICG) were calculated for each sample. Table 3 shows the results of ICG encapsulation in alginate/oligochitosan particles.

TABLE 3

| | Mass of | | |
|---|---|---|---|
| | trapped ICG | | |
| Sample | (mg) | % EE | % ICG |
| M10 | 0.0174 | 34.77 | 3.82 |
| M20 | 0.0484 | 48.39 | 9.96 |
| M30 | 0.0702 | 46.78 | 13.82 |
| M40 | 0.0888 | 44.38 | 16.87 |
| M50 | 0.2228 | 89.12 | 33.74 |

In contrast to the method described above, in the second encapsulation method during the gelation process an ICG solution was mixed with the oligochitosan solution and the mixture was subsequently added to the alginate as previously described. It was carried out according to the following method: (i) 200 μL of ICG solution (5 mg/mL) was added to 5 mL of oligochitosan (1 mg/mL); (ii) The mixture was added dropwise to a solution containing 2.5 mL of alginate (1.5 mg/mL) and 2.5 mL of deionized water; (iii) The particles were centrifuged at 15000 rpm for 15 min at 10° C.; (iv) The concentration of ICG in the supernatant was determined and therefore the mass of untrapped ICG; (v) The mass of trapped ICG, % EE and % ICG were calculated. Table 4 shows the results obtained from the encapsulation of ICG in the alginate/oligochitosan particles.

TABLE 4

| Mass of | | |
|---|---|---|
| trapped ICG | | |
| (mg) | % EE | % ICG |
| 0.9820 | 98.20 | 7.27 |

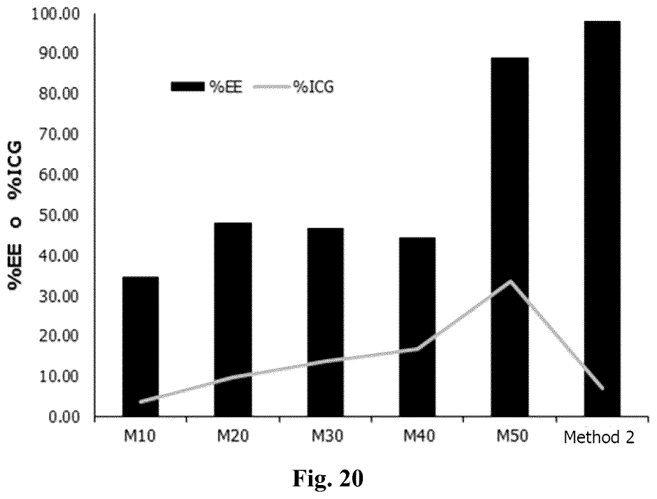

FIG. 20 shows a comparison of the encapsulation efficiency (% EE) and the percentage loading obtained with the two methods for the functionalization of ICG in biodegradable oligochitosan/alginate colloidal particles. It is important to emphasize that, although in the first method (i) higher percentages of encapsulation efficiency and trapped ICG mass were obtained, the release kinetics are most likely very different in the samples prepared by the two methods. A much faster release would be expected in the samples prepared by the first method (i), since the ICG is probably adsorbed on the surface, while the release could be slower in the sample loaded with the second method 2, where the ICG would be expected to be in more internal regions of the particles.

In accordance with the above, it can be seen that the system for the release of bioactive compounds comprising biodegradable colloidal particles has been designed for application in the pharmaceutical and biotechnology industry, and it will be evident to any expert in the field that the embodiments of the invention as described above and illustrated in the accompanying drawings are only illustrative but not limiting of the present invention, since numerous significant changes are possible in its details without departing from the scope of the invention. For example, it is possible to use the method described for obtaining biodegradable colloidal particles and use different biopolymers to those shown in the previously described examples.

Therefore, the present invention should not be considered as restricted except as required by the prior art and by the scope of the appended claims.

1. A system for the release of bioactive compounds, the system comprising biodegradable colloidal particles with an average size of less than 500 nm, wherein the biodegradable colloidal particles comprise an anionic polysaccharide, a cationic polysaccharide, and at least one bioactive compound.

2. (canceled)

3. The system according to claim 1, wherein at least one of:
the anionic polysaccharide is alginate;
the cationic polysaccharide is selected from chitosan, oligochitosan, or mixtures; or
a combination thereof.

4. The system according to claim 3, wherein the cationic polysaccharide is oligochitosan.

5. The system according to claim 1, wherein the bioactive compounds have antitumor activity and are photosensitizing.

6. A method for obtaining biodegradable colloidal particles, the method comprising: (a) estimating the stoichiometric charge ratio between an anionic polysaccharide based on its degree of deesterification, and a cationic polysaccharide based on its degree of deacetylation; (b) preparing a solution with the anionic polysaccharide based on the stoichiometric charge ratio and adjusting to an acidic pH; (c) preparing a solution with the cationic polysaccharide based on the stoichiometric charge ratio and adjusting to an acidic pH; (d) adding the solution with the cationic polysaccharide to the solution with the anionic polysaccharide to obtain biodegradable colloidal particles; (e) reducing the particle size of the biodegradable colloidal particles; (f) separating the biodegradable colloidal particles from the supernatant; and (g) resuspending the biodegradable colloidal particles and storing them.

7. (canceled)

8. The method according to claim 6, wherein at least one of:
the anionic polysaccharide is alginate;
the cationic polysaccharide is selected from chitosan, oligochitosan or mixtures; or
a combination thereof.

9. The method according to claim 8, wherein the cationic polysaccharide is oligochitosan.

10. The method according to claim 6, wherein the solvent for preparing the polysaccharide solutions is water.

11. The method according to claim 6, wherein at least one of:
the degree of deesterification of step (a) is determined by potentiometric or conductometric titration;
the degree of deacetylation of step (a) is determined by nuclear magnetic resonance (NMR), UV-vis spectrophotometry, potentiometric or conductometric titration, or FTIR-ATR spectrophotometry; or
a combination thereof.

12. (canceled)

13. The method according to claim 6, wherein the solution of the anionic polysaccharide is adjusted to a pH of 4 or 5.

14. (canceled)

15. The method according to claim 6, wherein the solution of the cationic polysaccharide is filtered before use in step (d).

16. The method according to claim 6, wherein step (d) is performed by a constant drip technique with vigorous stirring.

17. The method according to claim 6, wherein the particle size reduction of step (e) is carried out by sonicating the biodegradable colloidal particles by placing the particles in a container that is located within ice water to mitigate overheating in the particles for periods of between 5 and 10 minutes.

18. The method according to claim 6, wherein the separation of step (f) is performed by centrifugation at a speed between 6000 and 7000 rpm.

19. The method according to claim 6, wherein the resuspension of step (g) is performed by vortexing for at least 10 minutes.

20. The method according to claim 6, wherein the method comprises an additional step of functionalizing the biodegradable colloidal particles with at least one bioactive compound.

21. The method according to claim 20, wherein the functionalization step is carried out prior to step (d), where the solution of the cationic polysaccharide obtained in step (e) is mixed with a solution with at least one bioactive compound.

22. The method according to claim 20, wherein the functionalization step is performed after step (g), which in turn comprises the steps of: (i) adding a solution with at least one bioactive compound to a container with the biodegradable colloidal particles; (ii) adding deionized water to the mixture; (iii) stirring; (iv) centrifuging; (v) resuspending the previously functionalized biodegradable colloidal particles with deionized water to eliminate the non-trapped bioactive compound; and (vi) lyophilizing the functionalized biodegradable colloidal particles.

23. The method according to claim 22, wherein at least one of:
the step of (iii) stirring is performed in a vortex for at least 1 hour;
the step of (iv) centrifuging is performed for between 9000 and 15000 rpm for between 15 and 30 minutes at a temperature of at least 10° C.; or
a combination thereof.

24. (canceled)

25. The method according to claim 22, wherein the bioactive compounds have antitumor activity and are photosensitizers.
